(19) United States
(12) Patent Application Publication
(10) Pub. No.: US 2026/0248475 A1
(43) Pub. Date: Aug. 27, 2026

(54) METHOD FOR GENERATING LUNG TOMOGRAPHIC IMAGE BASED ON LUNG SOUND, AND LUNG MONITORING SYSTEM USING THE METHOD

(71) Applicant: UIF (University Industry Foundation), Yonsei University, Seoul (KR)

(72) Inventors: Su Hwan LEE, Seoul (KR); Kyu Bom KIM, Seoul (KR); Kyu Seok KIM, Seoul (KR); Yeon Kyeong KIM, Seoul (KR); Chanyo WON, Seoul (KR)

(21) Appl. No.: 19/544,118

(22) Filed: Feb. 19, 2026

(30) Foreign Application Priority Data
Feb. 27, 2025 (KR) 10-2025-0025826

(51) Int. Cl. A61B 7/00; A61B 5/00; A61B 5/08; A61B 7/04

(52) U.S. Cl. CPC A61B 7/003; A61B 5/0803; A61B 5/7425; A61B 5/746; A61B 7/04

(57) ABSTRACT

A method for lung monitoring provided by a lung monitoring system, includes: obtaining respiratory intensity of lung sound signals measured at a chest of a patient; generating lung sound tomographic images divided into lung regions by displaying a respiratory intensity at the lung regions corresponding to a position where the lung sound signals are measured; calculating a peak compliance according to a variation in a positive end-expiratory pressure (PEEP) value for each lung region of the lung sound tomographic image; and determining an optimal PEEP range of the patient based on a PEEP value corresponding to the peak compliance for each lung region.

### CROSS-REFERENCE TO RELATED APPLICATION

This application claims priority to and the benefit of Korean Patent Application No. 10-2025-0025826 filed with the Korean Intellectual Property Office on Feb. 27, 2025, the entire contents of which are incorporated herein by reference.

### BACKGROUND

### (a) Field

The present disclosure relates to lung monitoring.

### (b) Description of the Related Art

The need for rapid diagnosis and real-time monitoring of respiratory and pulmonary diseases has recently emerged. In particular, in the case of a patient with Acute respiratory distress syndrome (ARDS), an inflammatory lung injury occurs due to various causes, and thus the patient uses a respirator. In this case, a Ventilator induced lung injury (VILI) may occur if the mechanical ventilation is not properly carried out. Therefore, an optimal Lung protective ventilation (LPV) strategy is needed for minimizing the ventilator induced lung injury.

In general, in order to determine an optimal lung protective ventilation strategy, a method for analyzing a clinical index such as a tidal volume, a compliance, a volume-pressure, an oxygensaturation (SpO₂), and the like while decreasing a positive end-expiratory pressure (PEEP) of a patient is used. However, there is a limitation in that abnormalities at the alveolar level or regional ventilation abnormalities are difficult to detect using only basic information provided by the ventilator, such as pressure, volume, and flow.

To address this limitation, technologies such as chest radiography (X-ray), computed tomography (CT), and electrical impedance tomography (EIT) have recently been used for lung monitoring. However, a chest X-ray image may have reduced contrast for a small lesion and a foreign substance in a process of displaying the 3-dimensional structure of the lung as a 2-dimensional superimposed image, and a human interpretation error may occur depending on a screen adjustment value for each image. In addition, a CT image involves a high level of radiation exposure and limited accessibility and thus it is difficult to apply for real-time monitoring. An EIT image has an advantage of enabling real-time lung monitoring, but it requires multiple electrodes to be attached to a patient's chest in order to continuously apply a small current, and its resolution is significantly lower than that of the CT or X-ray image, making accurate lung monitoring difficult.

Meanwhile, a lung sound or respiratory sound of the patient is also an important index for diagnose and predict a lung disease. However, the lung sound is unstructured data with unquantified characteristics for each respiratory disease, and when a medical staff directly auscultate the patient's lung sound, there may be a difference in a diagnostic result depending on the medical staff, or a problem with infection among the medical staff may arise. For this purpose, a non-contact lung sound measuring device is used, but since the conventional non-contact lung sound measuring device cannot measure a lung sound from multiple chest regions, the accuracy may be lower than that of the actual auscultation method.

### SUMMARY

The present disclosure relates to a lung sound-based lung tomographic image generation method and a lung monitoring system using the same.

The present disclosure generates a lung tomographic image that visualizes a lung cross-section in an image based on a multi-channel lung sound obtained from a plurality of chest regions, and provides a result of analyzing an optimal positive end-expiratory pressure (PEEP) using the generated lung tomographic image.

Some embodiments of the present disclosure provide a method for lung monitoring provided by a lung monitoring system, includes: obtaining respiratory intensity of lung sound signals measured at a chest of a patient; generating lung sound tomographic images divided into lung regions by displaying the respiratory intensity at the lung regions corresponding to a position where the lung sound signals are measured; calculating a peak compliance according to a variation in a positive end-expiratory pressure (PEEP) value for each lung region of the lung sound tomographic image; and determining an optimal PEEP range of the patient based on a PEEP value corresponding to the peak compliance for each lung region.

The generating the lung sound tomographic images may include: obtaining the lung sound signals from lung sound signal measurement patches attached to the patient's chest; obtaining a lung cross-section template corresponding to a position and a number of the attached patches; and displaying the respiratory intensity distribution of the lung sound signals measured at the corresponding position in at lung regions included in the lung cross-section template.

The displaying may further include restoring a missing portion by performing inpainting on each of the lung regions in which the respiratory intensity distribution of the lung sound signals is displayed.

The respiratory intensity may be at least one of a peak value, an effective value, a minimum-maximum average, a peak vector, and a rise-fall time obtained in a time domain of the lung sound signal, or at least one of an amplitude, a phase, and a period obtained in a frequency domain of the lung sound signal.

The generating the lung sound tomographic images may include: generating the lung sound tomographic images at a specific time point by using the lung sound signals obtained at regular time intervals; and generating continuous time-series lung sound tomographic images by sequentially generating the lung sound tomographic image at the specific time point.

The calculating the peak compliance may include: setting a region of interest (ROI) for each lung region of the lung sound tomographic images; and calculating the peak compliance for each lung region based on an amount of change in the respiratory intensity of the ROI according to a variation in the positive end-expiratory pressure value.

The amount of change in the respiratory intensity may be a value calculated based on an amount of change in brightness of pixels within the ROI.

The determining the optimal PEEP range may include: obtaining respiration related indexes of the patient; and adjusting the optimal PEEP range by analyzing a change in the at least one respiration related index.

The determining the optimal PEEP range may further include providing a lung injury notification to an external terminal based on a result of comparing a PEEP value set for the patient with the optimal PEEP range.

Another embodiment of the present disclosure provide a lung monitoring system including a memory and a processor configured to execute instructions stored in the memory, wherein the processor is configured to: obtain a respiratory intensity of lung sound signals measured at a chest of a patient; generate lung sound tomographic images divided into lung regions by displaying the respiratory intensity at the lung regions corresponding to a position where the lung sound signals are measured; calculate a peak compliance according to a variation in a positive end-expiratory pressure (PEEP) value for each lung region of the lung sound tomographic image; and determine an optimal PEEP range of the patient based on a PEEP value corresponding to the peak compliance for each lung region.

The processor may obtain the lung sound signals from lung sound signal measurement patches attached to the patient's chest; obtain a lung cross-section template corresponding to a position and a number of the attached patches; and display the respiratory intensity distribution of the lung sound signals measured at the corresponding position in at lung regions included in the lung cross-section template.

The processor may restore a missing portion by performing inpainting on each of the lung regions in which the respiratory intensity distribution of the lung sound signals is displayed.

The processor may generate the lung sound tomographic image at a specific time point by using the lung sound signals obtained at regular time intervals, and may generate continuous time-series lung sound tomographic images by sequentially generating the lung sound tomographic image at the specific time point.

The processor may set a region of interest (ROI) for each lung region of the lung sound tomographic image, and calculate the peak compliance for each lung region based on an amount of change in the respiratory intensity of the ROI according to a variation in the positive end-expiratory pressure value, and the amount of change in the respiratory intensity may be a value calculated based on an amount of change in brightness of pixels within the ROI.

The processor may obtain respiration related indexes of the patient, and adjust the optimal PEEP range by analyzing a change in the at least one respiration related index.

The processor may provide a lung injury notification to an external terminal based on a result of comparing a PEEP value set for the patient with the optimal PEEP range.

Another embodiment of the present disclosure provide a method for generating lung sound tomographic images provided by a lung monitoring system, the method including: obtaining lung sound signals from lung sound signal measurement patches attached to patient's chest; obtaining a lung cross-section template corresponding to a position and a number of the attached patches; and generating the lung sound tomographic images by displaying a respiratory intensity distribution of the lung sound signals measured at the corresponding position in at lung regions included in the lung cross-section template.

The generating the lung sound tomographic images may include restoring a missing portion by performing inpainting on each of the lung regions in which the respiratory intensity distribution of the lung sound signals is displayed.

The respiratory intensity may be at least one of a peak value, an effective value, a minimum-maximum average, a peak vector, and a rise-fall time obtained in a time domain of the lung sound signal, or at least one of an amplitude, a phase, and a period obtained in a frequency domain of the lung sound signal.

The generating the lung sound tomographic images may include: generating the lung sound tomographic image at a specific time point by using the lung sound signals obtained at regular time intervals; and generating continuous time-series lung sound tomographic images by sequentially generating the lung sound tomographic image at the specific time point.

According to the present disclosure, it is possible to simultaneously provide pathophysiological information through a patient's lung sound and anatomical information through a lung sound tomography image to a medical staff.

According to the present disclosure, since longitudinal analysis is possible by synthesizing the patient's lung sound and the lung sound tomography image, a medical staff can perform lung monitoring and lung disease diagnosis more accurately compared to single data.

According to the present disclosure, a lung-protective ventilation strategy suitable for a patient can be established by providing optimal positive end-expiratory pressure through a lung sound tomography image.

According to the present disclosure, since a change in the lung can be monitored in real time, lung injury that may occur during mechanical ventilation can be prevented.

### BRIEF DESCRIPTION OF THE DRAWINGS

FIG. 1 shows a schematic conceptual view of a lung monitoring system according to an embodiment.

FIG. 2 shows a configuration diagram of a lung monitoring system according to an embodiment.

FIG. 3 shows description of a lung cross-section template according to an embodiment.

FIG. 4 shows a description of a method for generating a lung sound tomographic image according to an embodiment.

FIG. 5 shows an example of a lung sound tomographic image generated according to an embodiment.

FIG. 6 shows a description of a method for analyzing an optimal PEEP to an embodiment.

FIG. 7 shows a flowchart of a lung monitoring method according to an embodiment.

FIG. 8 shows a flowchart of a method for generating a lung sound tomographic image according to an embodiment.

FIG. 9 shows a flowchart of a PEEP analysis method according to an embodiment.

FIG. 10 shows a hardware configuration diagram of a lung monitoring system according to an embodiment.

### DETAILED DESCRIPTION OF THE EMBODIMENTS

Hereinafter, with reference to the accompanying drawings, embodiments of the present disclosure will be described in detail so that those skilled in the art to which the present disclosure pertains can easily carry out the same. However, the present disclosure may be implemented in many different forms and is not limited to the embodiments described herein. Accordingly, the drawings and description are to be regarded as illustrative in nature and not restrictive. Like reference numerals designate like elements throughout the specification.

In addition, unless explicitly described to the contrary, the word “comprise”, and variations such as “comprises” or “comprising”, will be understood to imply the inclusion of stated elements but not the exclusion of any other elements. In addition, terms such as “ . . . unit”, “ . . . device”, “ . . . module”, and the like described in the specification mean a unit that processes at least one function or operation, which may be implemented by hardware or software, or a combination of hardware and software.

In the present disclosure, devices are composed of hardware including at least one processor, a memory device, a communication device, and the like, and a program that is executed in combination with the hardware is stored in a designated place. The hardware has a configuration and performance capable of executing the method of the present invention. The program includes instructions for implementing the operating method of the present invention described with reference to the drawings, and is executed in combination with hardware such as a processor and a memory device.

In the present disclosure, “transmission or providing” may include not only direct transmission or providing but also indirect transmission or providing through another device or using a bypass path.

In the present disclosure, expressions described in the singular may be interpreted as singular or plural unless an explicit expression such as “one” or “single” is used.

In the present disclosure, the same reference numerals refer to the same components regardless of the drawings, and “and/or” includes each and all combinations of one or more of the mentioned components.

In the present disclosure, terms including ordinal numbers such as first, second, etc., may be used to describe various components, but the components are not limited by the terms. The terms are used only for the purpose of distinguishing one component from another component. For example, a first component may be named a second component, and similarly, a second component may also be named a first component, without departing from the scope of rights of the present disclosure.

In the flowchart described with reference to the drawings in the present disclosure, the operation sequence may be changed, several operations may be merged, a certain operation may be divided, and a specific operation may not be performed.

In the present disclosure, lung sound tomography refers to imaged data obtained by acquiring a distribution of lung sound intensity for each lung region using multi-channel lung sound signals measured in a plurality of chest regions, and reconstructing the acquired distribution of lung sound intensity for each lung region into a lung cross-sectional shape. In addition, the lung sound tomographic image may be a plurality of datasets generated at regular time intervals.

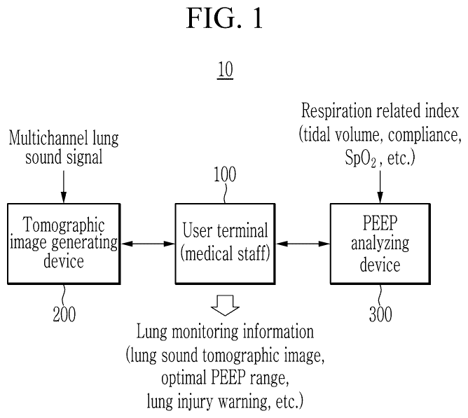

FIG. 1 shows a conceptual diagram of a lung monitoring system according to an embodiment.

Referring to FIG. 1, a method for generating a lung sound-based lung tomographic image and a lung monitoring system 10 (simply, a lung monitoring system) using the method is a computing device operating by at least one process, and includes a memory storing instructions and a processor executing the instructions, and operation of the present disclosure is performed as the processor executes instructions included in a computer program.

The lung monitoring system 10 may include a user terminal 100, tomographic image generating device 200, and a PEEP analysis device 300. In this case, the user terminal 100 is a terminal used by a medical staff performing lung monitoring, and may be a system or an external terminal connected to a hospital through a network. For example, the user terminal 100 may be a smart phone, a personal computer, and the like, but this is not restrictive, and may be any device that can provide lung monitoring information.

The lung monitoring system 10 may be implemented to interface with an external medical system and an external medical device. For example, the lung monitoring system 10 may receive multi-channel lung sound signal from an external lung sound measurement system (not shown), and may obtain a respiration related index such as a positive end-expiratory pressure (PEEP), a tidal volume, compliance, a saturation of percutaneous oxygen (SpO₂), an arterial oxygen partial pressure (PaO₂), a fraction of inspired oxygen concentration (FiO₂), and the like from an external monitoring device (not shown) such as a respirator.

Here, the multi-channel lung sound signal may be a lung sound signal measured in a plurality of chest regions of a patient using a plurality of wired (or wireless) patches attached to an anterior and posterior of the patient. In this case, the position to which the patch is attached and the number of patches may be changed according to determination of the medical staff, and the shape of a lung sound tomographic image may be changed according to the position to which the patch is attached and the number of patches.

Specifically, the tomographic image generating device 200 may generate a lung sound tomographic image from the received multi-channel lung sound signal, and the PEEP analysis device 300 may analyze an optimal PEEP range based on the received respiration related indexes.

In addition, the user terminal 100 may provide lung monitoring information including the lung sound tomographic image received from tomographic image generating device 200 and the optimal PEEP range received from the PEEP analysis device 300. In this case, the user terminal 100 may provide a lung injury warning notification when is a difference between the optimal PEEP range received from the PEEP analysis device 300 and a current PEEP value is greater than a predetermined threshold value.

Next, a lung monitoring information providing method for preventing lung injury by generating a lung sound tomographic image based on a multi-channel lung sound signal using the lung monitoring system 10, and analyzing the optimal PEEP range based on a lung sound tomographic image and respiration related indexes will be described in detail.

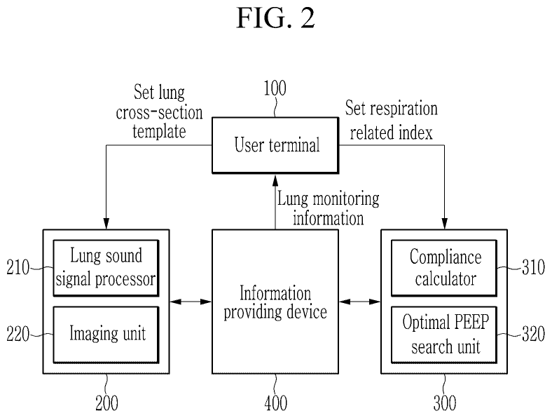

FIG. 2 shows a schematic diagram of a lung monitoring system according to an embodiment.

Referring to FIG. 2, the lung monitoring system 10 may include a user terminal 100, a tomographic image generating device 200, and a PEEP analysis device 300, and may further include an information providing device 400 for integrating various types of lung monitoring information and providing the integrated information to the user terminal 100.

In the present disclosure, the user terminal 100, the tomographic image generating device 200, the PEEP analysis device 300, and the information providing device 400 are named and referred to, but they are computing devices operated by at least one processor. In addition, interface between devices configurating the lung monitoring system 10 may be variously changed. For example, each device may be implemented in one computing device, or the devices may be distributed to separate computing devices. When the user terminal 100, the tomographic image generating device 200, the PEEP analysis device 300, and the information providing device 400 are implemented in a dispersed manner on separate computing devices, each device may communicate with each other remotely through a communication interface.

The tomographic image generating device 200 may include a lung sound signal processor 210 preprocessing a multi-channel lung sound signal and an imaging unit 220 that generates a lung sound tomographic image based on the preprocessed lung sound signal.

The lung sound signal processor 210 may receive a multi-channel lung sound signal measured in a plurality of chest regions of the patient from an external lung sound measurement system (not shown), and may remove a noise other than the lung sound such as a cardiac sound and the like from the received multi-channel lung sound signal. For example, the lung sound signal processor 210 may perform preprocessing using known methods such as resampling, normalization, noise reduction, and the like.

The imaging unit 220 may generate a lung sound tomographic image that represents the intensity of the preprocessed multi-channel lung sound signal in a 2-dimensional distribution form. For example, the intensity of the multi-channel lung sound signal may be one of a peak value, an effective value, a minimum-maximum average, a rise-fall time, and a peak vector obtained from a time domain of the lung sound signal or an amplitude, a phase, and a period obtained from a frequency domain. In the present disclosure, the intensity of the lung sound signal is described with an amplitude value example obtained from the time domain of the lung sound signal, but this is not restrictive.

The imaging unit 220 may receive a lung cross-section template from the user terminal 100, or may set an appropriate lung cross-section template based on lung sound measurement related information included in the multi-channel lung sound signal. For example, the lung sound measurement related information may be a position of a lung sound signal measurement patch attached to the chest of the patient or the number of lung sound signal measurement patches.

When an appropriate lung cross-section template is set, the imaging unit 220 may represent the lung sound signal with a different level of brightness according to the intensity. For example, the imaging unit 220 may extract a value indicating the intensity of the lung sound signal from the lung sound signal, and normalize the extracted value, and convert the normalized intensity into a Gaussian distribution form. After that, the imaging unit 220 may map the lung sound intensity converted into a Gaussian distribution onto a two-dimensional space to express the distribution of lung sound intensity differently depending on a level of brightness. For example, as the lung sound intensity is high, it may be represented with a dark pixel, and as the lung sound intensity is low, it may be represented with a bright pixel.

After that, the imaging unit 220 may generate a lung sound tomographic image by displaying the lung sound intensity expressed differently according to brightness in a region corresponding to a lung cross-section template. For example, lung sound intensity obtained from patch #1 may be disposed in region #1 of the lung cross-section template, and lung sound intensity obtained from patch #2 may be disposed in region #2 of the lung cross-section template.

Meanwhile, there may exist a missing portion in each region depending on the shape of the lung cross-section template. For this, the imaging unit 220 may restore the missing portion by performing inpainting. For example, the imaging unit 220 may perform inpainting using a known inpainting method such as diffusion-based inpainting, example-based inpainting, and the like, or a trained deep learning based inpainting model may be used. The inpainting method is an already known method, and therefore a detailed description will be omitted in the present disclosure.

As such, the imaging unit 220 may generate a lung sound tomographic image for a specific time point based on the lung sound intensity. However, in general, the lung sound signal may be a time-series type continuous signal. Therefore, the imaging unit 220 may repeatedly generate the lung sound tomographic image for a specific time point at regular time intervals, and may ultimately generate a plurality of lung sound tomographic images in a continuous time-series form. In this case, the time interval for generating the lung sound tomographic image may vary according to a determination of a medical staff, and each lung sound tomographic image may include time information indicating a time at which the image is generated.

The PEEP analysis device 300 may receive lung sound tomographic images in the time-series form generated by the tomographic image generating device 200, and may obtain an optimal PEEP range by analyzing the received lung sound tomographic images.

For this, the PEEP analysis device 300 may include a compliance calculator 310 that calculates a compliance for each lung region, and an optimal PEEP search unit 320 that searches for an optimal PEEP range based on the compliance. Here, the compliance is a parameter related to a lung injury such as overdistension and atelectasis, which may occur during mechanical ventilation, and the PEEP analysis device 300 may search for an optimal PEEP range based on the compliance.

The compliance calculator 310 may set at least one region of interest (ROI) for each of the plurality of lung regions included in the lung sound tomographic image, and may calculate compliance for each set ROI.

Meanwhile, compliance related to lung elasticity may be calculated based on a tidal volume and a driving pressure. In this case, in the case of the tidal volume, it may be estimated with lung sound intensity shown in the lung sound tomographic image. For example, the lung sound tomographic image is represented with different brightness according to the lung sound intensity, and therefore, the tidal volume may be estimated using the change in brightness of pixels within the corresponding ROI. In addition, the driving pressure may be calculated based on a different in alveolar pressure between inspiration and expiration. In this case, considering that the alveolar pressure and proximal airway pressure are in equilibrium when a flow reaches zero during mechanical ventilation, a driving pressure may be calculated using a plateau pressure and a PEEP value.

Therefore, the compliance calculator 310 can calculate the compliance using Equation 1.

$$Compliance = \frac{Δ V}{Plateau pressue - PEEP} \qquad ( Equation 1 )$$

Here, ΔV denotes a brightness change amount of piels in the ROI.

The compliance calculator 310 may calculate a compliance while gradually reducing a PEEP from a current set PEEP value. That is, the compliance calculator 310 may calculate a compliance according to the decrease of PEEP for each ROI.

The optimal PEEP search unit 320 may search for an optimal PEEP range based on the compliance according to the decrease of PEEP obtained for each ROI. For example, the optimal PEEP search unit 320 may determine a point where the compliance reaches the peak as the optimal compliance, and may determine an optimal PEEP range based on the optimal compliance for each ROI.

In addition, the optimal PEEP search unit 320 may adjust the optimal PEEP range by additionally considering the respiration related index. In this case, the respiration related index may include a tidal volume, compliance, a saturation of percutaneous oxygen (SpO₂), an arterial oxygen partial pressure (PaO₂), a fraction of inspired oxygen concentration (FiO₂), and the like from an external monitoring device (not shown) such as a respirator.

For example, the optimal PEEP search unit 320 may adjust the optimal PEEP range by analyzing the respiration related index in the case of actually applying the obtained optimal PEEP range. That is, when a specific respiration related index exceeds a predetermined threshold value even though it is included in the optimal PEEP range, the optimal PEEP range may be adjusted by excluding the corresponding PEEP value from the optimal PEEP range.

Finally, the information providing device 400 may transmit lung monitoring information including the plurality of lung sound tomographic images in time-series form and the optimal PEEP range to the user terminal 100. In addition, the user terminal 100 may provide the received lung monitoring information through a user interface screen.

The information providing device 400 may provide a compliance graph for each ROI according to the decrease of PEEP and a respiration related index change analysis result according to the optimal PEEP value. For example, when medical staff selects compliance at a specific time point on the compliance graph through the user interface, the information providing device 400 may provide the lung sound tomographic image and information on the ROI corresponding to the corresponding time point. In this case, the information providing device 400 may visually present the corresponding lung region by overlaying it in the form of a heat map on the lung sound tomographic image.

In addition, the information providing device 400 may provide a lung injury warning notification through the user terminal 100 when the obtained optimal PEEP range and the current PEEP value differed by more than the predetermined threshold range.

In this manner, medical staff may implement an effective lung protective ventilation (LPV) strategy based on the patient's lung monitoring information provided through the user terminal 100, thereby minimizing the occurrence of ventilator induced lung injury (VILI).

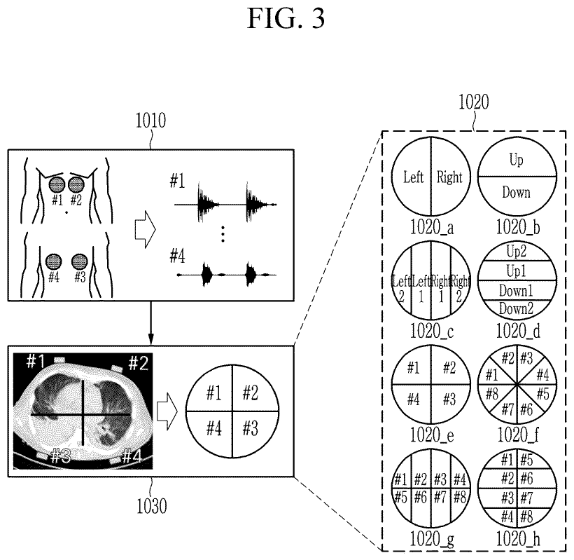

FIG. 3 shows description of a lung cross-section template according to an embodiment.

Referring to FIG. 3, the lung sound signal processor 210 may obtain multi-channel lung sound signals measured from lung sound measurement patches #1 to #4 (1010). That is, the lung sound signal processor 210 may obtain as many lung sound signals as many as the number of the lung sound signal measurement patches.

The imaging unit 220 may receive the lung cross-section template from the user terminal 100, or set an appropriate lung cross-section template based on lung sound measurement related information included in the multi-channel lung sound signal (1020). For example, when two patches #1 and #2 are attached to the anterior chest and two patches #3 and #4 are attached to the posterior chest to measure a multi-channel lung sound signal, the imaging unit 220 may select 1020_e template based on the lung sound measurement related information (1030).

Here, the lung cross-section template 1020 may include templates 1020_a and 1020_c in which patches are attached to the left and right sides of the chest, a 1020_b in which one patch is attached to each of the anterior chest and the posterior chest, a 1020_d template in which patches are attached to top, middle, and bottom portion of the chest, and templates 1020_e, 1020_f, 1020_g, and 1020_h in which a plurality of patches are attached to the anterior, posterior, and lateral portions of the chest. In addition, if necessary, the medical staff may additionally add a required lung cross-section template 1020 through a user interface of the user terminal 100.

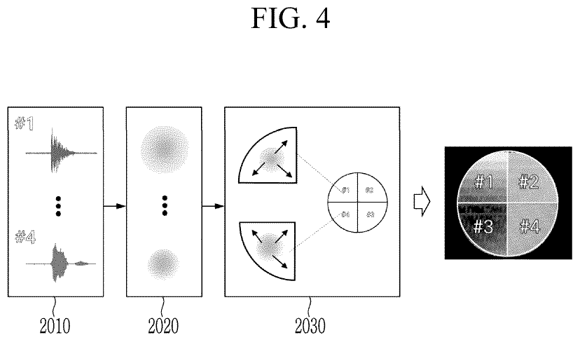

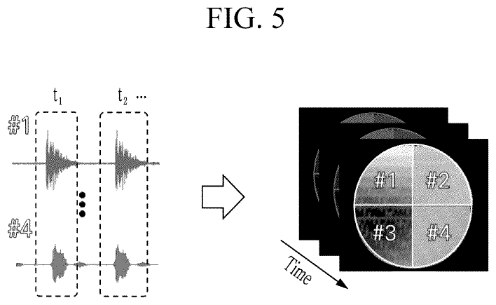

FIG. 4 shows a description of a method for generating a lung sound tomographic image according to an embodiment, and FIG. 5 shows an example of a lung sound tomographic image generated according to an embodiment.

A multi-channel lung sound signal measured by attaching two patches #1 and #2 to the anterior chest and attaching two patches #3 and #4 to the posterior chest will be exemplarily described.

Referring to FIG. 4, the lung sound signal processor 210 may obtain a plurality of lung sound signals from the lung sound signal measurement patches #1 to #4 attached to the patient's chest (2010). In this case, the imaging unit 220 may select a 1020_e template corresponding to the lung sound signal based on the lung sound measurement related information included in the lung sound signal.

The imaging unit 220 may extract and normalize a value representing lung sound signal intensity from each of the lung sound signals (#1-#4), and may convert the normalized lung sound intensity to a Gaussian distribution form. In addition, the imaging unit 220 may map the lung sound intensity, converted into a Gaussian distribution, onto a 2-dimensional space and represent the distribution of the lung sound intensity according to a level of brightness (2020).

The imaging unit 220 may generate a lung sound tomographic image for a specific time point by displaying the lung sound intensity obtained from each of the lung sound signals (#1 to #4) to regions (#1 to #4) corresponding to the lung cross-section template. In this case, the imaging unit 220 may restore a missing portion by performing inpainting if the missing portion exists in the lung region (2030).

In this manner, the imaging unit 220 may repeatedly generate a lung sound tomographic image for a specific time point at predetermined time intervals (t₁ to tₙ), and ultimately generate continuous time-series sound tomographic images as illustrate in FIG. 5.

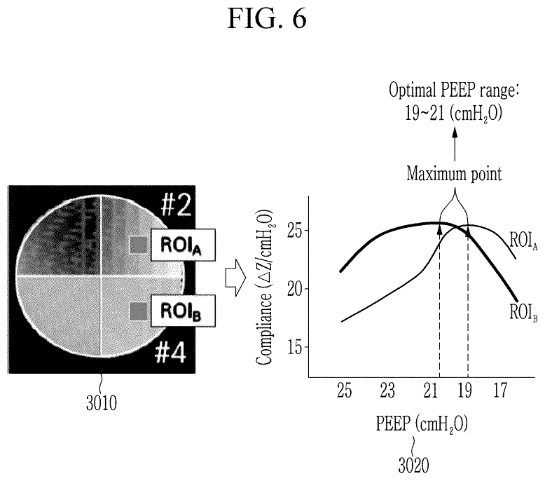

FIG. 6 shows a description of a method for analyzing an optimal PEEP.

Referring to FIG. 6, the compliance calculator 310 may set at least one ROI for each of a plurality of lung regions included in the lung sound tomographic image, and may calculate a compliance for each ROI (3010). Here, it is exemplarily illustrated that ROI A (ROI_{A}) is set in the region #2 of the lung cross-section template and ROI B (ROI_{B}) is set in the region #4.

The compliance calculator 310 may calculate the compliance for each ROI while gradually decreasing a PEEP from a currently set PEEP value (3020). For example, the compliance calculator 310 may calculate the compliance while the PEEP value is gradually decreased by 2 cmH₂O increments from 25 cmH₂O until it reaches 17 cmH₂O.

The optimal PEEP search unit 320 may obtain the optimal PEEP range based on the compliance according to the decrease of PEEP obtained for each ROI. For example, when the optimal compliance in the ROI A (ROI_{A}) is 19 cmH₂O and the optimal compliance in the ROI B (ROI_{B}) is 21 cmH₂O, the optimal PEEP search unit 320 may determine 19-21 cmH₂O as the optimal PEEP.

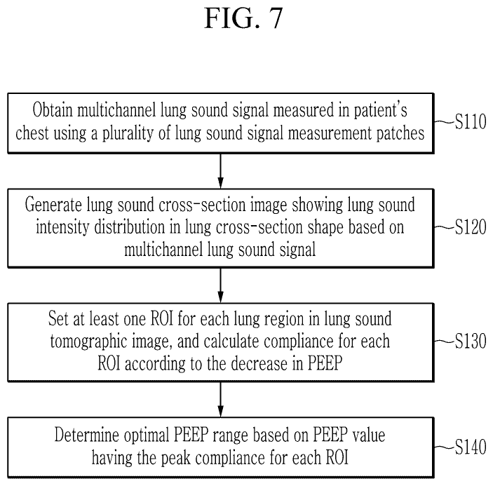

FIG. 7 shows a flowchart of a lung monitoring method according to an embodiment.

Referring to FIG. 7, the lung monitoring system 10 obtain a multi-channel lung sound signal measured from the patient's chest using a plurality of lung sound signal measurement patches (S110). In this case, a position to which a lung sound signal measurement patch is attached and the number of lung sound signal measurement patches may vary depending on the medical staff's judgement, and the shape of the lung sound tomographic image may vary according to the attached position and the number of patches.

The lung monitoring system 10 generates a lung sound tomographic image that shows a lung sound intensity in the shape of lung cross-section based on the multi-channel lung sound signal (S120). For example, the lung monitoring system 10 may extract and normalize the intensity of lung sound signal, convert the normalized lung sound intensity into a Gaussian distribution form, and map the Gaussian-distributed lung sound intensity with varying levels of brightness.

The lung monitoring system 10 sets at least one ROI for each lung region in the lung sound tomographic image, and calculates compliance for each ROI according to the decrease in the PEEP (S130). In this case, the lung monitoring system 10 may calculate a compliance while gradually decreasing the PEEP from the currently set PEEP value.

The lung monitoring system 10 determines an optimal PEEP range based on a PEEP value having the maximum compliance for each ROI (S140). In addition, the lung monitoring system 10 may provide a lung injury warning notification through the user terminal 100 when the optimal PEEP range differs from the current PEEP value by more than the predetermined threshold range.

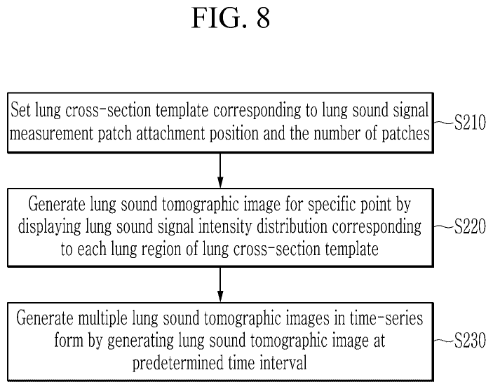

FIG. 8 shows a flow chart of a method for generating a lung sound tomographic image according to an embodiment.

Referring to FIG. 8, the lung monitoring system 10 sets a lung cross-section template corresponding to a position to which the lung sound signal measurement patch is attached and the number of patches (S210). For example, the lung monitoring system 10 may receive a lung cross-section template from the user terminal 100, or may set an appropriate lung cross-section template based on lung sound measurement related information included in the multi-channel lung sound signal.

The lung monitoring system 10 generates a lung sound tomographic image for a specific time point based on the lung sound intensity by displaying distribution of a lung sound signal intensity corresponding to each lung region of the lung cross-section template (S220). In this case, the lung monitoring system 10 may restore a missing portion in each lung region by performing inpainting.

The lung monitoring system 10 generates a plurality of lung sound tomographic images by generating a lung sound tomographic image for a specific time point at a constant time interval (S230). In this case, a lung sound tomographic image generated at each point may include time information, and a time interval for generation of the lung sound tomographic image may vary depending on the judgement of the medical staff.

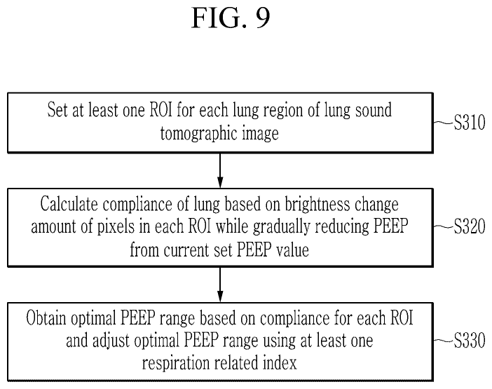

FIG. 9 shows a flowchart of a PEEP analysis method according to an embodiment.

Referring to FIG. 9, the lung monitoring system 10 sets at least one ROI for each lung region of the lung sound tomographic image (S310).

The lung monitoring system 10 calculates a compliance of the lung based on a brightness change amount of pixels in each ROI while gradually decreasing the PEEP from a current PEEP value (S320). In this case, the lung monitoring system 10 may estimate a tidal volume using the brightness change amount of the pixels in the ROI, and may calculate a compliance based on the tidal volume and a driving pressure (Plateau-PEEP).

The lung monitoring system 10 obtains an optimal PEEP range based on a compliance for each ROI, and adjusts the optimal PEEP range using at least one respiration related index (S330). For example, the lung monitoring system 10 may obtain a respiration related index including a tidal volume, compliance, a saturation of percutaneous oxygen (SpO₂), an arterial oxygen partial pressure (PaO₂), a fraction of inspired oxygen concentration (FiO₂), and the like from an external monitoring system such as a respirator, and may adjust the optimal PEEP range by analyzing the respiration related index in the case of actually applying the obtained optimal PEEP range.

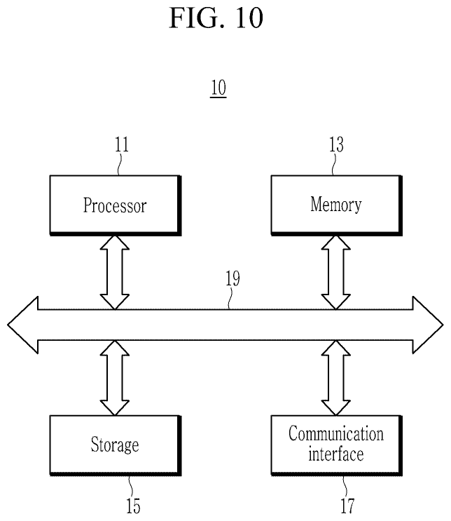

FIG. 10 shows a hardware configuration diagram of a lung monitoring system according to an embodiment.

Referring to FIG. 10, the lung monitoring system 10 is a computing device operating by one or more processors 11, and may include a processor 11, a memory 13 that loads a computer program executed by the processor 11, a storage 15 that stores the computer program and various types of data, a communication interface 17, and a but 19 connecting them. In addition to the stated configurations, various configurations may further be included in the lung monitoring system 10.

The computer program may include instructions that causes the processor 13 to execute a method/operation according to various embodiments of the present disclosure when being loaded to the memory 13. That is, the processor 11 executes the instructions such that methods/operations according to various embodiments of the present disclosure can be performed. The computer program refers to a series of computer-readable instructions grouped by function and executed by a processor. The computer program may include instructions that perform the operation described in the present disclosure.

The processor 11 controls the overall operation of each configuration of the lung monitoring system 10. The processor 11 may include at least one of a central processing unit (CPU), a micro processor unit (MPU), a micro controller unit (MCU), a graphics processing unit (GPU), or any other type of processor well known in the technical field of the present disclosure. In addition, the processor 11 may perform an operation for at least one application or computer program to execute the method/operation according to various embodiments of the present disclosure.

The memory 13 stores various types of data, instruction, and/or information. The memory 13 may load one or more computer programs from the storage 15 in order to execute the method/operation according to various embodiments of the present disclosure. The memory 13 may be implemented as volatile memory such as a RAM. However, the technical scope of the present disclosure is not limited thereto.

The storage 15 may store the computer program in a non-transitory manner. The storage 15 may include non-colatile memory such as a read-only memory (ROM), an erasable programmable ROM (EPROM), an electrically erasable programmable ROM (EEPROM), a non-volatile memory such as a flash memory and the like, a hard disk, a removable disk, or any other type of computer-readable recording medium well known in the technical field to which the present disclosure pertains.

The communication interface 17 supports wireless/wired internet communication of the lung monitoring system 10. In addition, the communication interface 17 may support various communication methods other than Internet communication. For this purpose, the communication interface 17 may include a communication module well known in the technical field of the present disclosure.

The bus 19 provides a communication function between configurations of the lung monitoring system 10. The bus 19 may be implemented in various forms, including an address bus, a data bus, a control but, and the like.

The embodiment of the present disclosure described above is not implemented only through the apparatus and method, but may also be implemented through a program that realizes a function corresponding to the configuration of the embodiment of the present disclosure or a recording medium in which the program is recorded.

Although the embodiment of the present disclosure has been described in detail above, the scope of the present disclosure is not limited thereto, and various variations and improvements of a person of ordinary skill in the art using the basic concept of the present disclosure defined in the following claims also fall within the scope of the present disclosure.

What is claimed is:

1. A method for lung monitoring provided by a lung monitoring system, comprising:
obtaining respiratory intensity of lung sound signals measured at a chest of a patient;
generating lung sound tomographic images divided into lung regions by displaying the respiratory intensity at the lung regions corresponding to a position where the lung sound signals are measured;
calculating a peak compliance according to a variation in a positive end-expiratory pressure (PEEP) value for each lung region of the lung sound tomographic image; and
determining an optimal PEEP range of the patient based on a PEEP value corresponding to the peak compliance for each lung region.

2. The method of claim 1, wherein the generating the lung sound tomographic images comprises:
obtaining the lung sound signals from lung sound signal measurement patches attached to the patient's chest;
obtaining a lung cross-section template corresponding to a position and a number of the attached patches; and
displaying the respiratory intensity distribution of the lung sound signals measured at the corresponding position in at lung regions included in the lung cross-section template.

3. The method of claim 2, wherein the displaying further comprises
restoring a missing portion by performing inpainting on each of the lung regions in which the respiratory intensity distribution of the lung sound signals is displayed.

4. The method of claim 1, wherein the respiratory intensity is
at least one of a peak value, an effective value, a minimum-maximum average, a peak vector, and a rise-fall time obtained in a time domain of the lung sound signal, or
at least one of an amplitude, a phase, and a period obtained in a frequency domain of the lung sound signal.

5. The method of claim 1, wherein the generating the lung sound tomographic images comprises:
generating the lung sound tomographic images at a specific time point by using the lung sound signals obtained at regular time intervals; and
generating continuous time-series lung sound tomographic images by sequentially generating the lung sound tomographic image at the specific time point.

6. The method of claim 1, wherein the calculating the peak compliance comprises:
setting a region of interest (ROI) for each lung region of the lung sound tomographic images; and
calculating the peak compliance for each lung region based on an amount of change in the respiratory intensity of the ROI according to a variation in the positive end-expiratory pressure value.

7. The method of claim 6, wherein the amount of change in the respiratory intensity is a value calculated based on an amount of change in brightness of pixels within the ROI.

8. The method of claim 1, wherein the determining the optimal PEEP range comprises:
obtaining respiration related indexes of the patient; and
adjusting the optimal PEEP range by analyzing a change in the at least one respiration related index.

9. The method of claim 1, wherein the determining the optimal PEEP range further comprises
providing a lung injury notification to an external terminal based on a result of comparing a PEEP value set for the patient with the optimal PEEP range.

10. A lung monitoring system comprising:
a memory; and
a processor executing instructions stored in the memory,
wherein the processor is configured to:
obtain a respiratory intensity of lung sound signals measured at a chest of a patient;
generate lung sound tomographic images divided into lung regions by displaying the respiratory intensity at the lung regions corresponding to a position where the lung sound signals are measured;
calculate a peak compliance according to a variation in a positive end-expiratory pressure (PEEP) value for each lung region of the lung sound tomographic image; and
determine an optimal PEEP range of the patient based on a PEEP value corresponding to the peak compliance for each lung region.

11. The lung monitoring system of claim 10, wherein the processor is configured to:
obtain the lung sound signals from lung sound signal measurement patches attached to the patient's chest;
obtain a lung cross-section template corresponding to a position and a number of the attached patches; and
display the respiratory intensity distribution of the lung sound signals measured at the corresponding position in at lung regions included in the lung cross-section template.

12. The lung monitoring system of claim 11, wherein the processor is configured to restore a missing portion by performing inpainting on each of the lung regions in which the respiratory intensity distribution of the lung sound signals is displayed.

13. The lung monitoring system of claim 10, wherein the processor is configured to:
generate the lung sound tomographic image at a specific time point by using the lung sound signals obtained at regular time intervals; and
generate continuous time-series lung sound tomographic images by sequentially generating the lung sound tomographic image at the specific time point.

14. The lung monitoring system of claim 10, wherein the processor is configured to:
sets a region of interest (ROI) for each lung region of the lung sound tomographic image; and
calculate the peak compliance for each lung region based on an amount of change in the respiratory intensity of the ROI according to a variation in the positive end-expiratory pressure value; and
the amount of change in the respiratory intensity is a value calculated based on an amount of change in brightness of pixels within the ROI.

15. The lung monitoring system of claim 10, wherein the processor is configured to:
obtain respiration related indexes of the patient; and
adjust the optimal PEEP range by analyzing a change in the at least one respiration related index.

16. The lung monitoring system of claim 10, wherein the processor is configured to
provide a lung injury notification to an external terminal based on a result of comparing a PEEP value set for the patient with the optimal PEEP range.

17. A method for generating lung sound tomographic images provided by a lung monitoring system, comprising:
obtaining lung sound signals from lung sound signal measurement patches attached to patient's chest;
obtaining a lung cross-section template corresponding to a position and a number of the attached patches; and
generating the lung sound tomographic images by displaying a respiratory intensity distribution of the lung sound signals measured at the corresponding position in at lung regions included in the lung cross-section template.

18. The method of claim 17, wherein the generating the lung sound tomographic images comprises
restoring a missing portion by performing inpainting on each of the lung regions in which the respiratory intensity distribution of the lung sound signals is displayed.

19. The method of claim 17, wherein the respiratory intensity is
at least one of a peak value, an effective value, a minimum-maximum average, a peak vector, and a rise-fall time obtained in a time domain of the lung sound signal, or
at least one of an amplitude, a phase, and a period obtained in a frequency domain of the lung sound signal.

20. The method of claim 17, wherein the generating the lung sound tomographic images comprises:
generating the lung sound tomographic image at a specific time point by using the lung sound signals obtained at regular time intervals; and
generating continuous time-series lung sound tomographic images by sequentially generating the lung sound tomographic image at the specific time point.
